(19) United States
(12) Patent Application Publication
(10) Pub. No.: US 2023/0312436 A1
(43) Pub. Date: Oct. 5, 2023

(54) METHOD AND PROCESS ARRANGEMENT FOR PRODUCING HYDROCARBONS AND USE

(71) Applicant: TEKNOLOGIAN TUTKIMUSKESKUS VTT OY, Espoo (FI)

(72) Inventor: Matti NIEMINEN, VTT (FI)

(21) Appl. No.: 18/041,783

(22) PCT Filed: Aug. 19, 2021
(86) PCT No.: PCT/FI2021/050561

§ 371 Date: Feb. 15, 2023

(30) Foreign Application Priority Data
Aug. 20, 2020 (FI) 20205809

(51) Int. Cl. C07C 4/22; C10J 3/54; B01D 46/00; C10J 3/86

(52) U.S. Cl. CPC C07C 4/22; C10J 3/54; B01D 46/00; C10J 3/86; C10J 2300/0946; C10J 2300/0976; C10J 2300/1853

(57) ABSTRACT

The invention relates to a method and process arrangement for producing hydrocarbons from plastic containing raw material by means of a gasification process in which the plastic containing raw material is gasified with steam at low gasification temperature of 680-740° C. in a fluidized bed gasifier for forming a product mixture comprising hydrocarbons, and the residence time is below 4 s at the gasification temperature, and the product mixture is cooled rapidly after the gasification to temperature which is below 640° C. for slowing and/or stopping the gasification reaction of the plastic containing raw material in order to increase propylene yield of the gasification process. Further, the invention relates to the use of the method.

### FIELD

The application relates to a method defined in claim 1 and a process arrangement defined in claim 11 for producing hydrocarbons, e.g. olefins, such as ethylene and/or propylene. Further, the application relates to a use of the method defined in claim 15.

### BACKGROUND

Known from the prior art is to produce hydrocarbons and olefins from fossil raw material by a cracking process. Further, it is known from the prior art that plastic waste may be treated to form energy or products. However, plastic waste is difficult to recycled and to utilize as raw material in new products.

From EP 0544840 is known a method for recovering ethylene from a polymeric waste material, wherein the polymeric waste material is heated to a temperature of 650-1000° C. with a circulating heat transfer material in a CFB pyrolyzer at a rate between about 500-50000° C/s and the polymeric waste material, and the heat transfer material is fed in a cocurrent flow and the ratio of the heat transfer material to the polymeric waste material is about 100:1-1:1. The residence time is less than 2 seconds to recover said ethylene.

### OBJECTIVE

The objective is to disclose a new type method and process arrangement for producing hydrocarbons, such as olefins, from plastic containing raw material. Further, the objective is to disclose a method and process arrangement for improving product yield and product distribution. Further, the objective is to disclose a method and process arrangement for improving propylene yield. Further, the objective is to replace virgin fossil raw material in olefin production, e.g. in ethylene and/or propylene production.

### SUMMARY

The method and process arrangement and use are characterized by what are presented in the claims.

In the method and process arrangement, plastic containing raw material is gasified with steam at low gasification temperature in a fluidized bed gasifier for forming a product mixture, and the product mixture is cooled after the gasification.

### BRIEF DESCRIPTION OF THE DRAWINGS

The accompanying drawing, which is included to provide a further understanding of the invention and constitutes a part of this specification, illustrates some embodiments of the invention and together with the description help to explain the principles of the invention. In the drawing:

FIG. 1 is a flow chart illustration of a process according to one embodiment.

### DETAILED DESCRIPTION

In the method for producing hydrocarbons from the plastic containing raw material by means of a gasification process, the plastic containing raw material (1) is gasified with steam (3) at low gasification temperature of 680-740° C. in a fluidized bed gasifier (2) for forming a product mixture (4) comprising hydrocarbons, and the residence time is below 4 s at the gasification temperature, i.e. at the reaction temperature during the gasification in the gasifier, and the product mixture (4) is cooled rapidly, and immediately, after the gasification to temperature which is below 640° C. for slowing and/or stopping, such as at least for slowing down or even for stopping, the gasification reaction of the plastic containing raw material in order to increase propylene yield of the gasification process. Preferably, the product mixture is cooled rapidly and immediately after the gasification.

The process arrangement for producing hydrocarbons from the plastic containing raw material comprises a fluidized bed gasifier (2) in which the plastic containing raw material (1) is gasified with steam (3) at low gasification temperature of 680-740° C. and with the residence time of below 4 s at the gasification temperature for forming a product mixture (4) comprising hydrocarbons, and at least one cooling device for cooling the product mixture (4) rapidly, and immediately, after the gasification to temperature which is below 640° C. for slowing and/or stopping the gasification reaction of the plastic containing raw material in order to increase propylene yield of the gasification process.

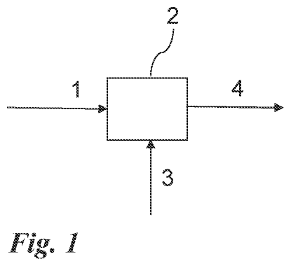

One embodiment of the method and the process arrangement is shown in FIG. 1.

In this context, the plastic containing raw material (1) means any material, which comprises at least plastic and/or polymer. The plastic containing raw material may consists of one or more raw material components. The plastic containing raw material may comprise waste or waste material. Further, the plastic containing raw material may comprise waste material and other material, e.g. any plastic material, recycled material, recovered fuel material or the like. In one embodiment, the plastic containing raw material comprises at least plastic containing waste, e.g. plastic waste. In one embodiment, the plastic containing raw material consists of the plastic containing waste. The plastic containing waste means any waste which comprises at least plastic and/or polymer. In one embodiment, the plastic containing raw material may comprise plastic material and at least one of recycled material, recovered fuel material or the like. Often the plastic containing raw material is a heterogenous material. In one embodiment, the plastic containing raw material comprises at least polyolefins, e.g. polyethylene and/or polypropylene. The plastic containing raw material may contain also other polymers. Further, the plastic containing raw material may contain also other components, such as paper, cardboard and/or aluminium material, e.g. aluminium foil. In one embodiment, the plastic containing raw material is a mixed raw material. In one embodiment, the plastic containing raw material comprises polyethylene, polypropylene, PVC, other plastics, other components and/or impurities. In one embodiment, the plastic containing raw material is in a solid form, e.g. in the form of shredded material. In one embodiment, the plastic containing raw material is treated before the supply into the feeder, for example by means of grinding, milling, chopping, shredding, or the like. In one embodiment, the plastic containing raw material is crushed and/or ground. In one embodiment, particle sizes of the plastic containing raw material can vary, for example between 0.1 mm-100 mm in flat form particles and for example 0.1 mm-50 mm, in one embodiment 0.1-25 mm, in spherical type particles.

In one embodiment, the plastic containing raw material is polymer-based waste. In this context, the polymer-based waste means any waste which comprises polymer or polymers, or which consists of one or more polymers. Often the polymer-based waste is a heterogenous material. In one embodiment, the polymer-based waste comprises at least polyolefins, e.g. polyethylene or polypropylene. The polymer-based waste may contain also other polymers. Further, the polymer-based waste may contain also other components, such as paper, cardboard and/or aluminium material. In one embodiment, the polymer-based waste is a mixed plastic material. In one embodiment, the polymer-based waste comprises multi-layer plastic packages and products. In one embodiment, the polymer-based waste comprises polyolefins over 80 w-%, preferably over 90 w-%. In one embodiment, the polymer-based waste comprises polyolefins below 80 w-%, in one embodiment below 70 w-%, in one embodiment below 60 w-% and in one embodiment below 50 w-%. In one embodiment, the polymer-based waste comprises PVC plastic, in one embodiment below 2 w-%. In one embodiment, the polymer-based waste is in a solid form, e.g. in the form of shredded material. In one embodiment, the polymer-based waste is treated before the supply into the gasifier, for example by means of grinding, milling, chopping, shredding, briquetting, pelletizing and/or by other suitable treatment.

In this context, the product mixture (4) means any product mixture, product composition or product, such as product gas, from the gasification and the gasifier (2), wherein the product mixture comprises at least hydrocarbons. Usually the product mixture is a mixture of hydrocarbons, and further may comprise other components and/or compounds. The product mixture comprises one or more components which may be, for example, gaseous components and/or vaporized components. In one embodiment, the product mixture may contain ethylene, propylene, butadiene, aromatic hydrocarbons and/or other hydrocarbons. In one embodiment, the product mixture comprises at least olefins. In one embodiment, the product mixture comprises at least ethylene and/or propylene. In one embodiment, the product mixture comprises at least ethylene and propylene. The product mixture may comprise also solid particles and/or water vapour.

In this context, the gasification in a gasifier means any gasification process by steam. The gasification is a process that converts plastic containing raw material into gaseous products, e.g. hydrocarbons, such as olefins. This is achieved by treating the raw material at suitable temperatures, and preferably with a controlled amount of steam.

In the fluidized bed gasifier (2) or gasification any suitable bed material can be used as the bed material in the fluidized bed. In one embodiment, the bed material is sand and/or calcium-containing bed material.

In one embodiment, the gasifier (2) is a bubbling fluidized bed gasifier (BFB gasifier). In one embodiment, the gasification is a bubbling fluidized bed gasification (BFB gasification). Also the plastic containing material comprising undesired components, e.g. aluminium foil, can be treated by gasifying in the bubbling fluidized bed gasifier. In one embodiment, the gasifier is a circulating fluidized bed gasifier (CFB gasifier).

In one embodiment, the gasification temperature, i.e. gasification reaction temperature, is 700-740° C. In one embodiment, the gasification temperature is 700-730° C.

In one embodiment, the gasification is carried out under atmospheric pressure or close atmospheric pressure in the gasifier (2). In one embodiment, the gasification is carried out at a pressure of below 4 bar, in one embodiment below 3 bar and in one embodiment below 2 bar.

In one embodiment, the residence time is below 3.5 seconds at the gasification temperature. In one embodiment, the residence time is below 3.0 seconds at the gasification temperature. In one embodiment, the residence time is 2-3.5 seconds at the gasification temperature. In one embodiment, the residence time is 2-4 seconds, in one embodiment 2-3 seconds, at the gasification temperature.

Preferably, the product mixture (4) is cooled immediately after the gasification (2). The product mixture is cooled rapidly after the gasification. In one embodiment, the product mixture is cooled to temperature which is below 600° C., in one embodiment below 550° C. In one embodiment, the product mixture is cooled to temperature of 400-600° C. In one embodiment, the product mixture is cooled to temperature of 400-550° C., in one embodiment 400-500° C. In one embodiment, the product mixture is cooled to temperature of 600-640° C. In one embodiment, the product mixture is cooled to the desired temperature in a time period of below 2 seconds. Preferably, the product mixture is cooled by means of a cooling device. In one embodiment, the process arrangement comprises at least one cooling device for cooling the product mixture (4). In one embodiment, the process arrangement comprises more than one cooling device for cooling the product mixture (4). In one embodiment, the cooling device is selected from a heat exchanger, water quench or other suitable cooling device. In one embodiment, the cooling device is the heat exchanger and/or water quench. In one embodiment, the cooling device is the heat exchanger. In one embodiment, the cooling device is a water quench. When the gasification reaction is slowing, such as slowing significantly, or stopping, yiels of methane, benzene and tar can be decreased and yield of propylene can be increased in the product mixture. Then, also yield of olefins can be increased.

In one embodiment, the product mixture is filtered after the cooling. In one embodiment, the product mixture is filtered by a hot gas filter. Alternatively, solid particles can be removed from the product mixture by any suitable filter, cyclone or other particle removal device. In one embodiment, the process arrangement comprises at least one filter, e.g. a hot gas filter or other suitable filter or filtration device, for filtering the product mixture. In the filtration solid components, such as solid particles or solid impurities, can be removed from the product mixture. In one embodiment, the process arrangement comprises at least one cyclone or other particle removal device.

In one embodiment, the process arrangement comprises at least one feed inlet for supplying the plastic containing raw material (1) into the gasifier (2). In one embodiment, the process arrangement comprises at least one outlet for discharging the product mixture (4) out from the gasifier (2) and/or from the cooling device. The feed inlet may be any suitable inlet known per se, e.g. pipe, port or the like. The outlet may be any suitable outlet known per se, e.g. pipe, outlet port or the like.

In one embodiment, the process arrangement comprises at least one feeding device for feeding the plastic containing raw material (1) to the gasifier (2). In this context, the feeding device can be any feeding device, feeder or other suitable device.

In one embodiment, the steam (3) is supplied to the gasifier (2), e.g. from a bottom or bottom part.

The method and process arrangement can be operated as a continuous process.

In one embodiment, the product mixture (4) comprises over 45 w-% olefins, such as ethylene and propylene, and in one embodiment over 50 w-%, after the cooling. In one embodiment, the propylene yield of the gasification process is over 15%-w, preferably over 18%-w, in one embodiment over 20%-w, after the cooling. In one embodiment, the product mixture which consists of hydrocarbons comprises over 15%-w propylene, in one embodiment over 18%-w propylene, and in one embodiment over 20%-w propylene, after the cooling.

In one embodiment, the product mixture (4) may be supplied to a desired process or to a desired process step in the recovery process of the hydrocarbons.

In one embodiment, the method and process arrangement are used and utilized in a production of hydrocarbons such as olefins, e.g. ethylene and/or propylene from the plastic containing raw material, e.g. from the plastic containing waste. The product mixture, such as hydrocarbons, can be used as such or can be supplied, for example, to an additional treatment stage, e.g. to a recovery stage in which desired fractions can be recovered, or to a desired process, such as a polymerization process, oxidation process, halogenation process, alkylation process or other chemical process, or to a production of plastic. For example, ethylene and propylene are building blocks for plastic products, petrochemical products and chemicals.

Thanks to the invention plastic containing raw material can be treated and utilized easily and effectively. The desired hydrocarbons, like olefins, can be produced, and olefin yield can be improved. Further, share of propylene can be increased, and methane, benzene and tar yield can be reduced. Especially, the yield of light olefins, such as ethylene and propylene, can be maximized.

The present invention provides an industrially applicable, simple and affordable way to produce hydrocarbons and especially olefins from the plastic containing raw material. Further, the method and process arrangement offer a possibility to treat the plastic-based waste easily, and energy- and cost-effectively, and the waste can be upgraded. The present invention provides flexible and economically feasible way to recycle waste polyolefins back to olefins. The method and process arrangement are easy and simple to realize in connection with production processes.

### EXAMPLES

FIG. 1 presents the method and process arrangement for producing hydrocarbons continuously from polymer-based waste.

The process of FIG. 1 comprises a bubbling fluidized bed gasifier (2) in which the polymer-based waste (1) is gasified with steam (3) at low gasification temperature which is 680-740° C., for example 700-730° C., for forming a product mixture (4). The steam is fed to the gasifier, from a bottom of the gasifier. The gasification is carried out under atmospheric pressure or close the atmospheric pressure in the gasifier (2), and the residence time is below 4 s, for example below 3.5 s, e.g. 2-3.5 or 2-3 s, at the gasification reaction temperature.

The product mixture (4) is cooled to temperature which is below 640° C., for example to temperature of 400-600° C., immediately and rapidly after the gasification. The process arrangement comprises at least one cooling device, for example a heat exchanger and/or water quench.

The product mixture (4) comprises at least ethylene and propylene. Further, the product mixture may comprise other hydrocarbons. Preferably, the product mixture comprises at least propylene. A desired hydrocarbon fraction or desired fractions may be separated from the cooled product mixture.

In one embodiment, the process arrangement may comprise at least one filtering device, e.g. a hot filter, for filtering the cooled product mixture (4) and for removing solid components from the cooled product mixture.

### Example 1

The gasification process was studied in a bench scale bubbling fluidized bed gasifier.

Polyolefin-rich plastic waste was used as a feed and steam was used as a gasification agent in the gasifier. Low gasification temperatures were used, and the temperature was between 710-740° C. during the gasification. The residence time was between 2.5-4 s at the gasification reaction temperature in the gasifier. A product gas was produced during the gasification in the gasifier. The product gas was cooled rapidly and immediately after the gasification to temperature 600-640° C. for slowing down and even for stopping the gasification reactions. Four test periods were carried out and analysed. Test results, such as yields of the product components, and residence time are shown in Table 1.

TABLE 1
Yield, g/kg feed (dry)

| | Test 1 | Test 2 | Test 3 | Test 4 |
|---|---|---|---|---|
| CO (28.01 g/mol) | 2.2 | 2.3 | 2.8 | 2.5 |
| CO2 (44.01 g/mol) | 5.9 | 6.6 | 3.8 | 2.6 |
| H2 (2.02 g/mol) | 5.1 | 5.3 | 5.2 | 4.9 |
| CH4 (16.05 g/mol) | 98.1 | 97.1 | 100.2 | 95.9 |
| C2H2 (26.04 g/mol) | 1.3 | 1.7 | 1.8 | 1.9 |
| C2H4 (28.06 g/mol) | 284.8 | 292.5 | 315.8 | 311.2 |
| C2H6 (30.08 g/mol) | 46.9 | 45.3 | 46.7 | 45.2 |
| C3H6 (42.09 g/mol) | 179.8 | 178.8 | 205.0 | 202.0 |
| Other hydrocarbons C3Hy + | 139.2 | 146.5 | 178.7 | 184.6 |
| C4Hy + C5Hy (e.g. C4H6, | | | | |
| 56.10 g/mol) | | | | |
| Sum of C2H4 + C3H6 | 464.6 | 471.3 | 520.8 | 513.2 |
| SUM | 763.4 | 776.1 | 859.9 | 850.9 |
| Benzene | 82.8 | 79.9 | 78.7 | 70.6 |
| Other tar components | 120.8 | 117.2 | 105.3 | 100.4 |
| Residence time at reaction | 4 | 3.1 | 2.5 | 2.5 |
| temperature, second | | | | |

The results were compared with comparative results, in which a product gas was formed from the plastic waste by gasifying in a bubbling fluidized bed gasifier with temperature of about 710° C. and with a residence time of 4-8 seconds. In this comparative gasification, yield of ethylene was about 30-35%-w and yield of propylene was about 8-9%-w, and yield of benzene was about 11-15%-w, yield of tar was 16-20%-w and yield of methane was 17-18%-w.

In the tests, it was observed that high olefin yield can be achieved, and especially the yield of propylene can be increased in the product gas, when residence time below 4 s was used. It was observed from the test results that the yield of ethylene was 28-32%-w and the yield of propylene was 18-20.5%-w, and the total yield of light olefins was 46.5-52.1%-w. The yield of benzene was 7-8%-w and the yield of other tar compounds was 10-12%-w. The product gas was surprisingly rich in propylene. Further, methane yield, benzene yield and tar yield can be decreased.

The devices and equipments of the process used in these examples are known per se in the art, and therefore they are not described in any more detail in this context.

The method and process arrangement are suitable in different embodiments for producing hydrocarbons, e.g. propylene, from different plastic containing raw material.

The invention is not limited merely to the examples referred to above; instead many variations are possible within the scope of the inventive idea defined by the claims.

1. A method for producing hydrocarbons from plastic containing raw material by means of a gasification process, wherein the method comprises
gasifying the plastic containing raw material with steam at low gasification temperature of 680-740° C. in a fluidized bed gasifier for forming a product mixture comprising hydrocarbons, and the residence time is below 4 s at the gasification temperature, and
cooling the product mixture rapidly after the gasification to temperature which is below 640° C. for slowing and/or stopping the gasification reaction of the plastic containing raw material in order to increase propylene yield of the gasification process.

2. The method according to claim 1, wherein the gasifier is a bubbling fluidized bed gasifier.

3. The method according to claim 2, wherein the propylene yield of the gasification process is over 15% w.

4. The method according to claim 1, wherein the gasification temperature is 700-730° C.

5. The method according to any one of claim 1, wherein the residence time is below 3.5 s at the gasification temperature.

6. The method according to claim 1, wherein the residence time is 2-3 s at the gasification temperature.

7. The method according to claim 1, wherein the product mixture is cooled to temperature which is 400-600° C.

8. The method according to claim 1, wherein the plastic containing raw material comprises at least polyolefins and other polymers.

9. The method according to claim 1, wherein solid particles are removed from the product mixture after the cooling.

10. The method according to any one of claim 1, wherein the product mixture comprises at least ethylene and propylene.

11. A process arrangement for producing hydrocarbons from plastic containing raw material by means of a gasification process, wherein the process arrangement comprises
a fluidized bed gasifier in which the plastic containing raw material is gasified with steam at low gasification temperature of 680-740° C. and with the residence time of below 4 s at the gasification temperature for forming a product mixture comprising hydrocarbons, and
at least one cooling device for cooling the product mixture rapidly after the gasification to temperature which is below 640° C. for slowing and/or stopping the gasification reaction of the plastic containing raw material in order to increase propylene yield of the gasification process.

12. The process arrangement according to claim 11, wherein the gasifier is a bubbling fluidized bed gasifier.

13. The process arrangement according to claim 11 wherein the cooling device is a heat exchanger and/or water quench.

14. The process arrangement according to claim 11 wherein the process arrangement comprises at least one hot gas filter for filtering the cooled product mixture.

15. A use of the method according to claim 1, wherein the method is used in a production of hydrocarbons from the plastic containing raw material.
